SEQUENCE LISTING (2 sequences)

SEQ ID NO: 1 - DNA, 9 bp, Unknown
  example sequence provided to explain binding of complementary DNA strings
ggtatcgta

SEQ ID NO: 2 - DNA, 9 bp, Unknown
  example sequence provided to explain binding of complementary DNA strings
ccatagcat

(19) United States
(12) Patent Application Publication
(10) Pub. No.: US 2018/0187187 A1
(43) Pub. Date: Jul. 5, 2018

(54) SELF-ASSEMBLING MOLECULAR NANOSYSTEM FOR TARGETED DNA AND GENE DELIVERY

(71) Applicant: Eric R. Henderson, Ankeny, IA (US)

(72) Inventor: Eric R. Henderson, Ankeny, IA (US)

(21) Appl. No.: 15/821,559

(22) Filed: Nov. 22, 2017

Related U.S. Application Data
(60) Provisional application No. 62/425,888, filed on Nov. 23, 2016.

(51) Int. Cl. C12N 15/11; C12N 9/22; C12N 9/96; C12N 15/90

(52) U.S. Cl. CPC C12N 15/11; C12N 9/22; C12N 2310/20; C12N 15/902; C12N 9/96

(57) ABSTRACT

Devices and methods of use for delivering genetic material to specific targets in living systems. The delivery methods involve employing a DNA-based nanosystem that includes genetic material containing the genes or the gene products to be delivered. The genetic material is architecturally altered and otherwise modified such that it is sufficiently robust for delivery but also accessible to the normal gene processing mechanisms extant in living systems.

### CROSS-REFERENCE TO RELATED APPLICATIONS

This application claims priority to U.S. Provisional Patent Application No. 62/425,888, filed on Nov. 23, 2016, the entirety of which is hereby incorporated herein by reference.

### FIELD OF THE INVENTION

The disclosure relates to methods and compositions for the use of DNA-based nanosystems for gene editing. More particularly, the invention relates to methods and compositions for generating and using DNA nanosystems for site-specific gene editing to treat and cure gene-based diseases and conditions.

### BACKGROUND

Gene editing technology creates unprecedented opportunities for genetic modification in living systems, including humans, to treat and cure genetic disease. A key requirement is the ability to deliver the gene-editing machinery to specific locations, tissues, cells or other desirable targets. The invention described here addresses this requirement.

The goal of improving health through genetic engineering is decades old but despite many efforts and a few marginal successes the technology for robust genetic manipulation to cure disease is just now emerging. A key component of the emerging technology is a gene editing system called CRISPR/Cas9 and variants thereof (Applications of CRISPR technologies in research and beyond, Barrangou R, Doudna JA. Nat Biotechnol. 2016 September 8:933-941. doi: 10.1038/nbt.3659). This system, originally evolving in bacteria to combat viral infection, has been recognized as an incredibly powerful approach to targeted gene modification. With this new technology comes immense responsibility to use it appropriately and within a high ethical standard.

Despite its inherent utility and promise, the CRISPR/Cas9 system, and for that matter all gene-editing approaches, will always be compromised by the absence of a methods for direct and efficient delivery of the system to the intended cellular and subcellular targets with minimal (ideally zero) collateral damage to unintended targets. One approach to overcoming this limitation is to deliver gene-editing machinery to living systems, including humans, that are just a few cells old (i.e., early embryos). Of course, this opens a Pandora's box of ethical and technological issues. Thus, this approach is not widely practiced and will certainly be extremely highly regulated in the foreseeable future. Other methods such as microinjection or site perfusion with DNA carriers are inefficient and prone to both missing all target cells as well as hitting off-target sites. (Kaminski, R., Bella, R., Yin, C., Otter, J., Ferrante, P., Gendelman, H. E., Li, H., Booze, R., Gordon, J., Hu, W., Khalili, K. (2016) Excision of HIV-1 DNA by gene editing: A proof-of-concept in vivo study. Gene Therapy 23(8-9), 696).

A preferred strategy is to create a technology for delivery of gene editing machinery directly to the cells of interest in developing and fully developed organisms. This is a formidable task since it requires a delivery method that can be applied systemically, exposed to billions of cells, and yet target only a tiny fraction of the organismal cellular population. This technology must be biocompatible, biostable, highly malleable (chemically and architecturally) and cost effective.

Thus a simple and robust device and method of use for delivering genetic material (and nucleic acids that do not carry genes) to specific cells is desirable. Further, it is desirable for the device and method of use to leverage the ability of DNA (and RNA) to be utilized as an engineering matrix and sculpted into virtually any shape by one of several known methods including but not limited to those disclosed in: DNA Origami (Rothemund P. (2006) Folding DNA to create nanoscale shapes and patterns. Nature. 440, 297-302; Patents and Patent Applications; U.S. Pat. No. 7,842,793, WO2012061719, US20130224859, DNA Canvas (Y. Ke, L. Ong, W. Shih, P. Yin. (2012) Three-dimensional structures self-assembled from DNA bricks. Science 338, 1177-1183, and U.S. Pat. No. 9,796,749) and DAEDALUS (Veneziano, R., Ratanalert, S., Zhang, K., Zhang, F., Yan, H., Chiu, W., and Bathe, M. (2016) Designer nanoscale DNA assemblies programmed from the top down. Science 352(6293) 1534). It is further desirable for the device and method of use to leverage the ability of DNA (and RNA) to be chemically modified (with, for example but not limited to cell-specific antibodies, aptamers, ligands, modifiers of surface chemistry, proteins, lipids and carbohydrates) to create nanoscale molecular delivery vehicles that will seek and destroy only those cells for which they are programmed to do so, and for the technology to be capable of being systemically deployed (for example, but not limited to, administration by injection into the blood or lymphatic systems) or directly applied to a physiological target (for example a tumor, tissue, organ or other defined biological entity).

### BRIEF SUMMARY

In accordance with one embodiment of the invention, a biostable nucleic acid-based nanosystem for targeted delivery of gene-editing molecular machinery in cell culture, ex vivo cell explants, and in vivo, and the genes for that machinery are provided. The nanosystem comprises folded or otherwise architecturally sculpted nucleic acid (usually DNA or RNA; here the term DNA is used to represent all classes of nucleic acid with which this invention may be practiced). The nanoscale constructs created this way may include, but do not require, a variety of surface modifying entities designed to facilitate delivery of molecular species including but not limited to genes, genes encoding guide sequences and enzymes involved in gene editing, gene-editing molecules and molecular ensembles to specific biological targets in vitro and in vivo. The nanosystem may also contain and deliver antibodies, aptamers, small molecules, drugs, and other therapeutic or otherwise bioactive materials. In some embodiments, the nucleic acid is resistant to degradation in the blood stream, lymphatic system, tissues and interstitial spaces due to its non-biological architecture. Further, it can be readily modified or “decorated” with recognition molecules such that the molecular carrier system interacts with specific targets (e.g., a specific subset of cells in a multicellular milieu in vivo and in vitro). Finally, the nucleic acid to be delivered can be contained as a core of the nanosystem with a peripheral shielding component comprised of nucleic acids and modifying entities, or it can constitute the entire nanosystem itself, that is, the genetic and molecular cargo to be delivered and utilized for gene-editing can be folded to render them biostable and give them access to cellular, cytoplasmic, and nuclear targets. Once at the target site (tissue, cell, cytoplasm or nucleoplasm) the cellular machinery responsible for normal processes consistent with maintenance of organic life (including but not limited to DNA replication, DNA transcription, nucleic acid ligation, nucleic acid scission, nucleic acid reconfiguration, resolution of Holiday structures, protein recycling, and protein synthesis) cause the protected and masked molecular information contained in the nanosystem to be utilized which results in the creation and manifestation of the genetic information therein embodied.

In some embodiments, the output of the delivered genes, the gene products, alter the genetic landscape at the target site, which results in mitigation or cure of disease. In some embodiments, the gene products enhance biological performance. In alternative embodiments, the gene products are designed to interfere with cell function when desirable, as would be the case in incapacitating cancer cells.

### BRIEF DESCRIPTION OF THE DRAWINGS

FIG. 1 illustrates a nanosystem architecture in accordance with an embodiment of the invention.

FIG. 2 illustrates a modified nanosystem architecture in accordance with an embodiment of the invention.

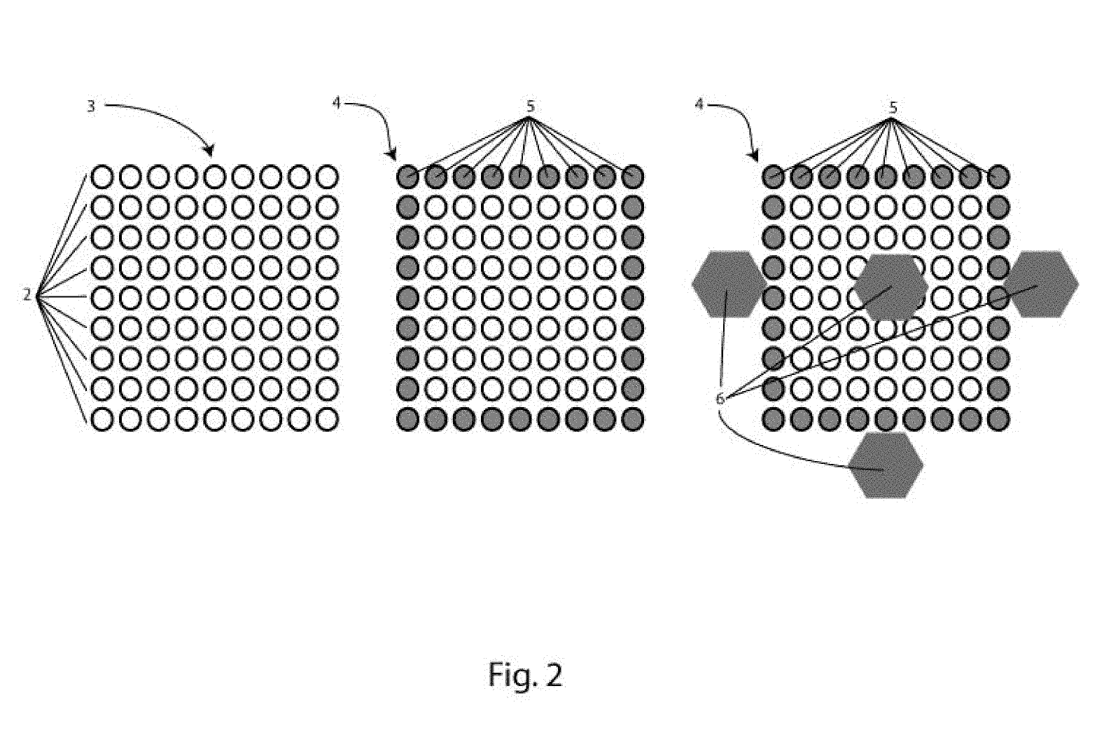

### DETAILED DESCRIPTION

Some embodiments of the present invention will now be described more fully hereinafter with reference to the accompanying drawings, in which some, but not all, embodiments of the invention are shown. Indeed, various embodiments of the invention may be embodied in many different forms and should not be construed as limited to the embodiments set forth herein; rather, these embodiments are provided so that this disclosure will satisfy applicable legal requirements. Like reference numerals refer to like elements throughout. Some components of the apparatus are not shown in one or more of the figures for clarity and to facilitate explanation of embodiments of the present invention.

DNA's Engineering Code

The systems and methods disclosed herein are enabled by a unique feature of nucleic acids (DNA and RNA and variants thereof). The term DNA is used to include all nucleic acids and variants thereof. The unique feature is the ability of the DNA base sequence to define precisely how two strands or two sections of a single strand of DNA will pair. These base pairing rules are the essence of the process of life on the planet and also define an architectural or “engineering” code for DNA.

DNA contains the iconic genetic code, which is centered upon the ability of the four common bases, adenine (A), guanine (G), cytosine (C) and thymine (T) (uracil (U) in RNA; it is understood that the features of DNA are also present in RNA to a great extent) to form “base pairs” in which G pairs with C and A pairs with T (J. D. Watson and F. H. C. Crick. (1953) A Structure for Deoxyribose Nucleic Acid (1) Nature (3), 171, 737-738).

Base pairing rules are: G-C, A-T, and A-U. These rules are extended to modified bases and any variants that allow specific pairing of the bases.

The genetic code is embodied in strings of bases (usually three at a time), which encode amino acids. The amino acids thus encoded constitute one form of genetic output and are strung together to create proteins comprised of specific amino acid sequences. Additionally, the order of the bases and base pairs in DNA act as markers for a variety of entities that act upon DNA and include, but are not limited to, transcription factors, histones, and enzymes. Further, each strand of DNA has a polarity (designated 5 prime, 5′; to 3 prime, 3′). The strands are usually antiparallel (though not exclusively so in all forms of pairings which include duplex, triplex, quadruple, pseudoknot and other forms). Typically if one strand runs 5′ to 3′, the complementary strand runs 3′ to 5′.

While this view of the function of DNA has been studied and used for myriad purposes since its discovery in 1953, a more advanced perception of DNA has resulted in the recognition of an inherent “engineering code” as mentioned above. Again, this code is present in all nucleic acids and variants thereof, including chemically altered, chiral (mirror image), DNA, RNA, and any modified nucleic acid derivative in which base pairing is an extant phenomena.

This engineering code, like the genetic code, is centered upon the ability of strings of DNA bases to bind by base pairing to precisely complementary strings but only with much less stability to partially complementary strings. Thus, for example, the string:

5′-GGTATCGTA-3′

binds with highest affinity to the precise complement,

3′-CCATAGCAT-5′

to form the duplex,

5′-GGTATCGTA-3′
|||||||||
3′-CCATAGCAT-5′

and with much lower affinity and bond strength to imperfect complements wherein one or more of the base pairs are mismatched. This characteristic of precise binding of one string of DNA bases to its exact complement allows one to use DNA as a material for creating structures and functional devices following the principle of nucleotide base pairing. Shapes and devices made using this DNA engineering code are referred to as DNA-nanosystems or nanosystems herein henceforth.

### Self-Assembly

A key feature of DNA nanosystems is that once the design has been determined, the parts can be mixed and they will spontaneously form the desired base pairs resulting in the formation of the desired nanosystem. The process of following a set of embedded self-encoded directions to create a specific architecture is referred to as “self-assembly”. Thus, DNA nanosystems self assemble; they do not require external information or user intervention once all the components have been mixed. The entire set of instructions for creating the desired nanosystem is already extant within the molecular ensemble constituting the mixture.

The thermodynamics of DNA base pairing does require that a chemical and thermal environment be created that is conducive to high yield of the desired nanosystem. The final nanosystem can be considered a preferable “low energy state” but to arrive at this state it is often necessary to kinetically stimulate the sample by heating to disentangle any undesirable metastable (stable transiently but not the lowest energy state) architectures. Thus, the sample is often heated to a temperature above the “melting temperature” (that temperature at which the DNA is fully single stranded) and then cooled (slowly or quickly depending upon the desired outcome) to produce a high yield of the desired self-assembled DNA nanosystem. Variations on this theme exist that, in some instances, can result in a much more rapid process for formation of the desired nanostructure, but in all cases the DNA is heated and cooled to allow it to assume the final desired architecture.

The solution in which the DNA is suspended has a significant impact upon the self-assembly process. While a vast number of variant conditions may be used, in general a monovalent and a divalent cation (usually chosen from the group of Na+, K+, Mg++) are present and a buffer, often Tris base brought to approximately pH 7 at room temperature with acetic acid (commonly referred to as Tris-acetate, pH 7) is used to maintain a neutral pH. Finally, a chemical that chelates (binds to and inactivates) divalent species is often present (Ethylenediaminetetraacetic 3 acid: EDTA) to minimize the activity of enzymes that digest DNA (DNAses), which often requires divalent cations. It is noteworthy that the Mg++ concentration usually exceeds the chelation threshold of the EDTA so there is free Mg++ to interact with, and to stabilize DNA double helices. These conditions are exemplary, and not intended to define the only conditions used or that will work to create DNA nanostructures and, therefore, should not limit the scope of the present disclosure.

### DNA Origami

A common strategy for building complex DNA assemblies is to use a process termed “DNA Origami”. (Rothemund P. (2006) Folding DNA to create nanoscale shapes and patterns. Nature. 440, 297-302). In this process, a large single-stranded DNA molecule (the “scaffold”, usually, but not necessarily, a genome obtained from the bacterial virus (bacteriophage) M13 or similar) is mixed with a large number (˜200 is typical, though this number is highly variable) of small synthetic DNA molecules that form double helices (dsDNA) connected by crossovers related to a junction observed in DNA recombination events (Holiday junction). The small synthetic DNA molecules are called “staples” or “helper strands”, and they tie together distal (nonadjacent) portions of the large single-stranded “scaffold” strand into the desired shape. The analogy to folding paper to create shapes (origami) led to the term DNA origami. It is noteworthy that processes for accomplishing this process without the M13 scaffold, entirely from small molecules, has been demonstrated (Mathur, D. and Henderson, E. (2013) Complex DNA Nanostructures from Oligonucleotide Ensembles, ACS Synthetic Biology, 2, 180-185) because this method eliminates the need for a single-stranded scaffold and, therefore, creates many more avenues to create DNA nanosystems and to build multiple devices in a single reaction. One can leave portions of the DNA single stranded (ssDNA) and in this way create a structure that has one conformation but will change conformation when the synthetic DNA complementary to the remaining single stranded regions are added. This ssDNA>dsDNA conversion is the basis for operation of the systems and methods used in biosensing DNA nanosystems due to the fact that such transitions generate forces (pushing or pulling) in a user-defined vector orientation (Mathur, D., and Henderson, E. R. (2016) Programmable DNA Nanosystem for Molecular Interrogation. Nature Scientific Reports, 6, No. 27413, doi:10.1038/srep27413). In other words, by adding DNA to convert ssDNA to dsDNA, the user can pull on specific molecular pairs and measure the forces holding them together.

### Other DNA Sculpting Strategies

### DNA Canvas

Peng and coworkers described a method for creating two-dimensional and three-dimensional shapes using rationally designed building blocks of DNA oligomeric ensembles (Y. Ke, L. Ong, W. Shih, P. Yin. (2012) Three-dimensional structures self-assembled from DNA bricks. Science 338, 1177-1183). This method was named the “DNA Canvas” method. In some embodiments this method may be used to practice the invention described herein. In those embodiments, however, the stands would be discontinuous and would have to be globally ligated in such a way as to permit transcription of the genes thus constructed. This is a laborious method fraught with a high probability of failure and, therefore, is not a preferred method for practicing the present invention.

### Daedalus

Bathe has described a method for assembling DNA nanostructures by a process akin to, but distinct from, DNA origami (Veneziano, R., Ratanalert, S., Zhang, K., Zhang, F., Yan, H., Chiu, W., and Bathe, M. (2016) Designer nanoscale DNA assemblies programmed from the top down. Science 352(6293) 1534). This method is named DADELUS. The DADELUS method results in the construction of geometric shapes with internal cavities. In some embodiments this method may be used to practice the current invention although it would be limited by inhibition of transcription of genes embodied therein by physical and kinetic barriers resulting from the architecture of the structures constructed this way. Therefore, DNA origami is the preferred method, but not the only method, for constructing the nanosystems required to practice the invention described herein.

### Key Advantages

Listed below are some of the key advantages that can be realized by the systems and methods of the invention. These advantages are provided as examples, and not intended to limit the scope of the present disclosure.

1. Low cost: Billions of molecular delivery systems are created for a few cents (US).
2. Field stability: DNA is extremely stable, which is why it is a key forensics marker and can be extracted from fossils. Furthermore, DNA is a biological molecule and, as such, is inherently more harmonious with biological systems such as is required for in vivo molecular (e.g., drug) delivery.
3. Chemical malleability: DNA is readily modified by a large number of molecules relevant to targeted molecular delivery in complex cellular and organismal environments.
4. Ease of implementation: The general recipe of “add water and salt, heat, cool, use” applies to the systems and methods of the invention, and can be implemented in virtually any environment including portable and resource-starved locales.
In summary, the self-assembling bionanosystem described herein is a platform technology for delivery of molecular species to specific cell types in living systems.

### EXAMPLES

The examples provided below are intended to illustrate of some features and applications of the systems and methods described herein, and are not intended to limit the scope of the present disclosure.

### Example 1. A Biostable Gene-Delivery Nanosystem

In this embodiment, the DNA that contains genetic information for the components of the CRISPR gene-editing system (guide RNAs, the nuclease Cas9 or other nuclease) is directly integrated into the DNA nanosystem. The DNA containing the gene-editing ensemble is part of the scaffold of the nanosystem. In some instances, it may be advantageous to encase the gene-containing scaffold portion inside of additional DNA such that a DNA shell protects the genes. Thus, the nanosystem may contain both gene-encoding sequences and non-coding or otherwise supporting sequences that facilitate formation and action of the nanosystem but do not directly participate in producing genetic output such as the Cas9 (or other DNase) enzyme or the guide RNAs.

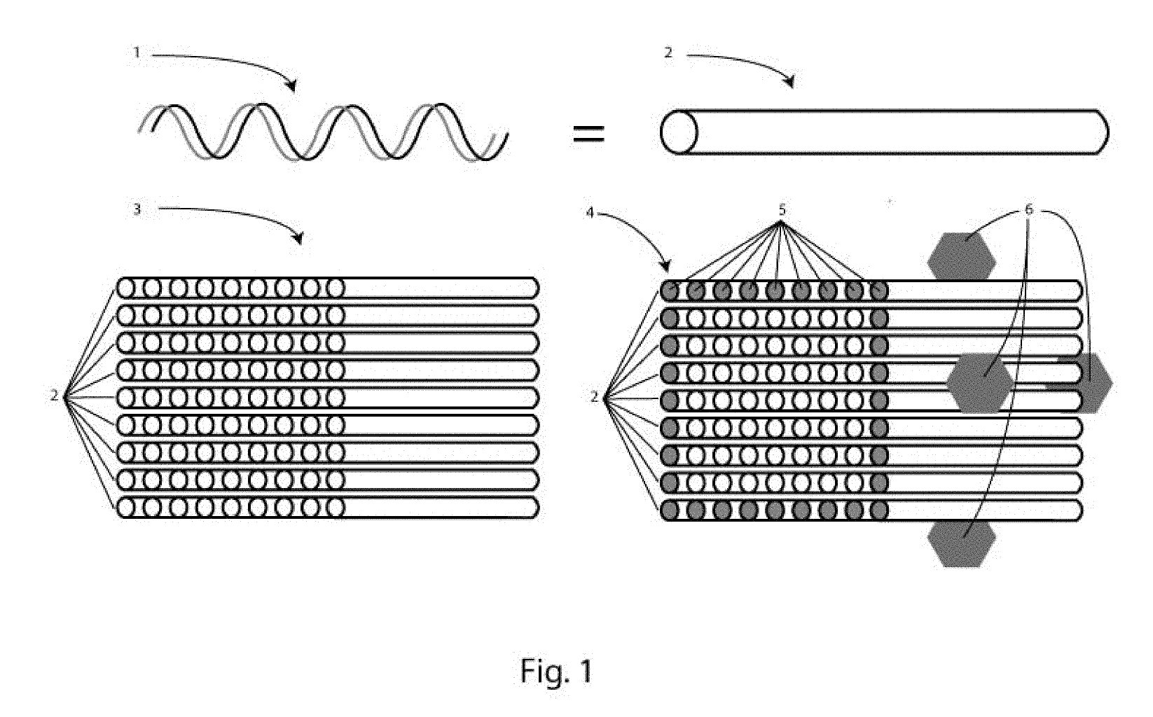

FIG. 1 depicts these principles. As depicted in FIG. 1 a DNA helix 1 is equated to a linear rod 2, which is used to facilitate illustration of the architecture of one embodiment of the invention. In this embodiment a nanosystem 3 comprises a 9×9 helix array that comprises 81 DNA helices 1, illustrated in FIG. 1 as linear rods 2, where each DNA helix 1 is about 73 base pairs long. The nanosystem 3 is generated by “folding” the gene-encoding DNA and ancillary DNA sequences into a defined structure using the technique of DNA origami. The 9×9 array is an arbitrarily selected shape for nanosystem 3 for illustrative purposes and not intended to limit in any way the possible architectural variants of the DNA nanosystem invention. Each helix 1 is tethered to an adjacent one by the phosphate backbone of the scaffold DNA (the end linkages are not shown in the figure for the sake of clarity). The external surface of the nanosystem 3 may be modified in several ways to create a modified DNA nanosystem 4. These modifications may serve several purposes including but not limited to, protection of the essential gene-encoding sequences, facilitation of transcription of the gene encoding sequences, bioavailability, survivability in a living system milieu, recognition of molecular/cellular targets, binding to molecular/cellular targets, cell membrane permeability, nuclear targeting, and nuclear membrane permeability. In one embodiment the modified nanosystem 4 may contain modified nucleotides 5 that are chemically distinct from biological DNA. Such chemical modifications may include, but are not limited to, phosphorothioate linkages that render the surface more hydrophobic than biological DNA and, therefore, increase the nanosystem's 4 compatibility with biological cellular and nuclear membranes. Additional modifications of the nanosystem 4 may include, but are not limited to, addition of a variety of molecular species including but not limited to proteins, lipids, enzymes, chemicals, materials, metals, aptamers, antigens, antibodies, or polymers to enhance function of the nanosystem. Native (unmodified) and modified DNA nanosystem 4 with an out shell of phosophorothioate 5 and protein 6 modified helices is illustrated in FIGS. 1 (orthographic three dimensional view) and 2 (end on view). Alternative and additional modifications are possible and those shown are intended to be exemplary but do not limit the scope of the invention with respect to modification of the nucleic architecture to enhance its function.

### Modification of the Gene Delivery Nanosystem to Enhance Function.

In many cases it is desirable to modify the DNA nanosystem to reduce (or enhance) inflammatory response, enhance longevity and structural integrity, facilitate cell recognition, and/or enhance cell penetration and targeting to specific cytoplasmic or nuclear locations. DNA is readily modified by a number of standard methods. For example, phosphorothioate inter-nucleotide linkages can be introduced to mitigate nuclease activity. Similarly, 2′O-methyl ribose modification stymie enzymatic degradation. Use of amino (NH3) terminated staple oligonucleotides provides a convenient method for addition of a vast array of molecular species (e.g., aptamers, antibodies, ligands and peptides) to the staple and thereby to the nanostructure of which they are an integral part. One non-limiting example is the used of N-hydroxysuccinimide chemistry to cross-link amino terminate moieties. Many other crosslinking methods exist as well, and may be readily employed by one skilled in the art. Further, in some instances chemicals, compounds, drugs, and pharmaceutical reagents may act as “intercalators”, that is, become sandwiched within the base pairs of the DNA-origami helices and thereby carried with the DNA-origami and modify its characteristics in a desirable fashion.

Examples of potential modifications are listed here in this non-exclusive list, and are not intended to limit the scope of the present disclosure in any way. These include:
Addition of ligands (biotin, small molecules)
Addition of proteins (e.g., antibodies, cell binding proteins)
Addition of chemical moieties (e.g., fatty acids, hydrophobic materials, amphipathic materials)
Addition of cellular signals (e.g., nuclear targeting protein sequences)
Addition of intercalators

### Example 2: Delivery of Gene-Editing Components to Cells Using a Biostable Gene Delivery Nanosystem

In this example, the nanosystem 3 or modified nanosystem 4 comprises a folded and thereby protected DNA-origami construct that includes genes and gene controlling elements for a gene editing protein (e.g., Cas9) and the associated guide RNA sequences that will target a specific sequence of interest in cells. Once the DNA-origami construct has passed through the plasma and/or nuclear membranes, the previously inert DNA components are recognized by standard DNA processing cellular machinery (including, but not limited to, polymerases, nucleases, ligases, helicases, repair enzymes, and/or cellular trafficking mechanisms). The genes in the DNA-origami are processed to create the Cas9 protein and guide RNA that assemble into an active gene-editing complex and carry out a desired gene-editing event.

### Example 3. In Vivo Rectification of Site-Specific Undesirable Genomic Information

In this example, the nanosystem 3 or modified nanosystem 4 comprises a DNA-origami nanosystem that is delivered to a cell, cells, organ, tissue or other biological entity, and the genetic cargo is converted by normal biological process into gene products. These gene products are capable of modifying genes in the target entity, and thereby rectify or otherwise alter the genetic environment (the genetic code of the target) such that its performance is altered. In most cases, this alteration is desirable including but not limited to removal, excision, inactivation, altering the nucleotide content, or rendering harmless of genes causing pathogenesis. For example, removal by excision of a latent viral gene or genome or modification of a latent viral gene or genome to mitigate viral outbreak. Latent viruses include but are not limited to herpes simplex virus (HSV) and human immunodeficiency virus (HIV). It is also possible to modify the genetic landscape to create harm as might be desirable in proactive destruction of cancer cells.

Many modifications and other embodiments of the invention will come to mind to one skilled in the art to which this invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1. A rationally designed nucleic acid-based nanosystem for transport and delivery of molecules and materials to biological targets comprising an array of helices containing genetic information.

2. The nanosystem of claim 1 wherein the nanosystem acts in vivo.

3. The nanosystem of claim 1 wherein the nanosystem acts in cell culture.

4. The nanosystem of claim 1 wherein the nanosystem acts ex vivo.

5. The nanosystem of claim 1 wherein the nanosystem is configured using DNA origami.

6. The nanosystem of claim 1 wherein the nanosystem is configured using DAEDALUS.

7. The nanosystem of claim 1 wherein the nanosystem is configured using DNA Canvas.

8. The nanosystem of claim 1 wherein the nanosystem further comprises supporting sequences.

9. The nanosystem of claim 1 further comprising non-coding elements.

10. The nanosystem of claim 9 further comprising a gene editing system.

11. The nanosystem of claim 10 wherein the gene editing system comprises CRISPR/Cas9.

12. The nanosystem of claim 9 further comprising one or more modified elements.

13. The nanosystem of claim 12 wherein the modified elements enhance the nanosystems function.

14. The nanosystem of claim 12 wherein the modified element comprises a molecular species.

15. A method of using a rationally designed nucleic acid-based nanosystem for transport and delivery of molecules and materials to biological targets for editing, enhancing, deleting, regulating, or modifying a genetic element in a living system comprising generating a nucleic acid-based nanosystem by folding one or more gene-encoding DNA and ancillary DNA sequences into a defined structure using a DNA sculpting method, and modifying the nanosystem with one or more modified nucleotides.

16. The method of claim 15 wherein the nanosystem is used to excise a genetic element.

17. The method of claim 15 wherein the nanosystem is used to insert a genetic element.

18. The method of claim 15 wherein the nanosystem is used to modify a genetic element.

19. The method of claim 15 wherein the nanosystem is used to inactivate latent virus genes.

20. The method of claim 15 wherein the nanosystem is used to excise a latent virus genome.
